(19) United States
(12) Patent Application Publication
(10) Pub. No.: US 2020/0155773 A1
(43) Pub. Date: May 21, 2020

(54) DETERMINATION OF AIR FLOW RATE THROUGH AN INHALER

(71) Applicant: Sensirion AG, Stäfa (CH)

(72) Inventors: Christoph ZIPKES, Stäfa (CH); Andreas ALT, Stäfa (CH)

(73) Assignee: Sensirion AG, Stäfa (CH)

(21) Appl. No.: 16/687,028

(22) Filed: Nov. 18, 2019

(30) Foreign Application Priority Data
Nov. 19, 2018 (EP) 18207022.7

(51) Int. Cl. A61M 15/00

(52) U.S. Cl. CPC A61M 15/0001; A61M 2016/0021; A61M 15/009

(57) ABSTRACT

An accessory (40) for an inhaler (10) comprises a fastening structure (42) for fastening the accessory to the inhaler. The accessory has a pressure port (43). When the accessory is fastened to the inhaler, the pressure port is arranged upstream from a mixing zone (33) of the inhaler with respect to an air flow (F1) caused by inhalation by a patient through the inhaler. An electronic sensor (45) is in communication with the pressure port. The electronic sensor is sensitive to a pressure change at the pressure port caused by the air flow.

### CROSS REFERENCE TO RELATED APPLICATIONS

This application claims priority to European Appl. No. 18207022.7, filed Nov. 19, 2018; the contents of which are incorporated by reference herein in their entirety.

### TECHNICAL FIELD

The present invention relates to an accessory for a medical inhaler, the accessory enabling the determination of an air flow rate through the inhaler. The invention further relates to an inhaler fitted with such an accessory, and to a method of determining an air flow rate through an inhaler using such an accessory.

### PRIOR ART

Medical inhalation devices (inhalers) are commonly used for delivering a drug in the form of an aerosol (i.e. a dispersion of fine solid particles or liquid droplets in air) to a patient's lungs. The most common types of inhalers are pressurized metered-dose inhalers (pMDIs) and dry-powder inhalers (DPIs).

A pMDI is disclosed, e.g., in US 2002/0144678 A1. The drug is stored in solution or suspension together with a propellant in a pressurized container. The container is closed by a metering valve. The container is held in a plastic housing defining an air inlet and an air outlet of the inhaler. At the air outlet, the housing forms a mouthpiece. The housing comprises a socket for receiving a valve stem of the metering valve. The socket defines a duct leading to a nozzle orifice located in the air flow path between the air inlet and the air outlet of the inhaler. In use, the patient inhales through the mouthpiece. This creates an air flow through the housing and past the nozzle orifice. After the patient has started inhaling, the patient depresses the container into the housing onto the valve stem, which is seated in the socket, thereby opening the metering valve and releasing a metered dose of the drug through the nozzle orifice. The released drug mixes with the air flow and is hence inhaled by the user through the mouthpiece. Usually, the complete pMDI is discarded after all the drug in the container has been consumed, i.e., the housing of the pMDI is normally not reused with a fresh drug container.

For optimum delivery of the drug, it is important that the patient, while inhaling, maintains an air flow in a certain desired range of flow rates, e.g., in a range of 30 to 60 Umin.

The creation of an air flow above a certain minimum flow rate or in a certain range of flow rates is also important for other types of inhalers. For instance, many dry-powder inhalers (DPIs) rely on the force of patient inhalation to break up the powder into particles that are small enough to reach the lung. It is therefore important that the patient maintains a sufficiently high air flow rate during inhalation.

There have been many suggestions in the prior art how patients can be trained to correctly create an air flow in a desired range of flow rates through an inhaler. F. Lavorini et al., “The ADMIT series—Issues in Inhalation Therapy. 6) Training tools for inhalation devices”, Primary Care Respiratory Journal 2010, 335-341, doi: 10.4104/perj.2010.00065 discloses various training tools to this end. However, most of these devices cannot be integrated into actual inhalers and can only be used for training.

GB 2490770 A discloses an adapter for fitting to the mouthpiece of a pMDI. The adapter is a tube with an air flow rate indicator, to be attached to the outlet end of the mouthpiece so as to be interposed between the pMDI and the patient's mouth. The document further discloses an inhaler with an integrated electronic flow rate sensor. The flow rate sensor is located inside the inhaler body just upstream of the outlet of the device. The adapter of this document is disadvantageous in that it prolongs the flow path between the point where the drug is injected into the air flow and the mouth of the user. The inhaler with integrated electronic flow rate sensor is disadvantageous in that the flow rate sensor cannot be reused when the inhaler is discarded, and in that the flow rate sensor necessarily modifies the air flow through the device.

### SUMMARY OF THE INVENTION

It is an object of the present invention to provide a device for determining an air flow rate through an inhaler, which can be reused and/or recycled separately from the inhaler, and which neither prolongs nor obstructs the aerosol flow through the inhaler downstream from the position where the drug is mixed with the air flow.

Accordingly, an accessory for an inhaler is provided, the inhaler having an air inlet for the entry of air into the inhaler and an air outlet for communication with the mouth of a patient, such that inhalation by a patient through the air outlet causes an air flow through the inhaler from the air inlet to the air outlet. The inhaler further defines a mixing zone between the air inlet and the air outlet for mixing the air flow with a drug. The accessory comprises a fastening structure for fastening the accessory to the inhaler and an electronic sensor for determining a flow rate of the air flow. The accessory comprises a pressure port, and the fastening structure is configured to fasten the accessory to the inhaler in such a manner that the pressure port is arranged upstream from the mixing zone with respect to the air flow. The electronic sensor is in communication with the pressure port and is sensitive to a pressure change at the pressure port caused by the air flow.

The present invention thus provides an attachment for an inhaler that enables a determination of the flow rate of the air flow through the inhaler. This allows direct conclusions about the manner in which the patient inhales the drug. Accordingly, feedback to the patient and/or information to medical personnel can be provided, based on the flow rate measurements by the accessory. The accessory is designed to be fastened to the outside of the inhaler, where it cannot disturb the aerosol flow. The pressure port of the attachment is arranged upstream from the point of injection of the drug into the air flow, where it cannot disturb the flow of the aerosol that is formed by mixing the released drug and the air flow. Since the flow of the aerosol is neither disturbed nor prolonged by the accessory, the accessory can be added to an existing inhaler without requiring new testing procedures or separate approval of the combination by health authorities. The accessory can be configured to be easily removable from the inhaler, such that it can be reused on a fresh inhaler and can be recycled separately from the inhaler once it has reached the end of its lifecycle.

The pressure port of the accessory is arranged such that an air flow that enters the inhaler through its air inlet causes a pressure change at the pressure port. The term “pressure port” is to be understood broadly as any structure that enables a direct or indirect determination of pressure at the location where the pressure port is situated, employing the electronic sensor. For instance, in some embodiments the pressure port can be formed by one or more openings that enable direct gas exchange with the electronic sensor. In this case, communication between the pressure port and the electronic sensor takes place pneumatically. In other embodiments, the pressure port can be formed by an elastic membrane that transmits force to the electronic sensor. In this case, communication between the pressure port and the electronic sensor takes place mechanically.

In particular, the accessory can be configured such that the air flow into the air inlet causes a pressure decrease (negative pressure) at the pressure port due to the flow velocity of the air flow. This effect is commonly called the Bernoulli effect or the Venturi effect.

In use of the accessory, the pressure port is located upstream from the mixing zone where the air flow and the drug are mixed to form an aerosol. Mixing of the air flow and the drug can be achieved, in particular, by releasing the drug into the air flow through an orifice, in particular, through a nozzle-type orifice, as it is the case in typical pMDIs. In this case, the mixing zone is a zone immediately downstream from the orifice. Accordingly, in this case the pressure port is advantageously arranged upstream from the orifice.

The inhaler can have an inhaler housing defining a receiving portion configured to receive a drug reservoir and an outlet portion forming a mouthpiece or being connected to a mouthpiece. The outlet portion and the receiving portion can extend at an angle to one another. Many pMDIs have such a configuration. The fastening structure can then be configured to fasten the accessory to the exterior of the receiving portion rather than to the exterior of the mouthpiece portion.

In some embodiments, the fastening structure is configured to fasten the accessory to the exterior of the inhaler in such a manner that the pressure port is arranged upstream from the air inlet of the inhaler with respect to the air flow. In this manner it is possible to retrofit an existing inhaler with an accessory for flow rate determination without modification of the existing inhaler.

The accessory can comprise an accessory housing, the electronic sensor being received in the accessory housing, and the pressure port can be formed by one or more openings in the accessory housing. In some embodiments, the pressure port is formed by a plurality of openings in the accessory housing, the openings being distributed circumferentially along a circumference of the air inlet of the inhaler across the direction of the air flow when the accessory is attached to the inhaler. In this manner, the total cross-sectional area of the pressure port can be increased without increasing the individual cross-sectional area of the individual openings, thereby reducing the risk of dust and liquids entering the housing through the openings and ensuring that the accessory remains functional even if one of the openings should get blocked.

In some embodiments, the pressure port is arranged in a wall portion of the accessory housing that is essentially flush with a housing wall of an inhaler housing when the accessory is fastened to the inhaler, the wall portion of the accessory housing and the housing wall of the inhaler housing delimiting a flow path of the air flow. In this manner, it can be ensured that the accessory will disturb the air flow that enters the inhaler only to the smallest possible degree.

More specifically, the accessory housing can define an axial stop structure configured to abut to a proximal end face of the housing wall when the accessory is fastened to the inhaler, and the wall portion in which the pressure port is arranged can be adjacent to the stop structure.

In some embodiments, the fastening structure comprises a ring configured to be arranged around an inhaler housing of the inhaler at the air inlet. In other embodiments, the fastening structure can comprise two prong-like arms that clasp the inhaler housing by the action of elastic forces. Many other ways of fastening the accessory to the inhaler housing are conceivable.

In some embodiments, the fastening structure is configured to fasten the accessory to an exterior of an inhaler housing of the inhaler, and the accessory is configured in such a manner that the pressure port is arranged in or behind a through-opening in a wall of the inhaler housing when the accessory is fastened to the inhaler. For instance, the pressure port can be formed by a pipe that extends into the through-opening, or it can be formed by an opening in a wall portion of the accessory housing, said opening being arranged behind the through-opening in the wall of the inhaler housing.

In some embodiments, the pressure port can be arranged such that the air flow overflows the pressure port when the accessory is fastened to the inhaler. In other embodiments, the pressure port can be arranged such that the pressure port is shielded from direct air flow, for instance by being arranged in a recess of the accessory adjacent to the air flow.

In some embodiments, the accessory is configured to modify the air flow adjacent to the pressure port in such a manner that a reverse air flow due to exhalation by the patient through the inhaler causes a pressure change at the pressure port with opposite sign as compared to the pressure change caused by the air flow due to inhalation. In this manner, the accessory can distinguish between inhalation and exhalation. To this end, the accessory can comprise a flow-modifying structure that causes a positive dynamic pressure at the pressure port only for one of the two flow directions. For instance, the flow-modifying structure can comprise an obstruction upstream from the pressure port with respect to the air flow created by inhalation and downstream from the pressure port with respect to the reverse air flow created by exhalation, thereby causing a dynamic pressure at the pressure port only when the reverse air flow is present.

The electronic sensor can be any type of sensor that is capable of detecting the pressure change at the pressure port caused by the air flow. For instance, the electronic sensor can be a pressure sensor, in particular, an absolute pressure sensor (measuring pressure in comparison to absolute vacuum) or a differential pressure sensor (measuring pressure relative to some reference pressure, in particular, relative to the ambient pressure). A differential pressure sensor is sometimes also called a relative pressure sensor. Many different types of pressure sensors are known, based on different working principles, and the present invention is not limited to a particular type of pressure sensor. For instance, some types of pressure sensors comprise a deformable membrane, and the degree of deformation of the membrane by the air pressure is determined. This working principle is often employed in barometric pressure sensors, and the pressure sensor can accordingly be a barometric pressure sensor.

In some embodiments, the electronic sensor is a differential pressure sensor or a flow sensor. Some types of differential pressure sensors in fact work on the principle that a pressure difference causes a small flow through a flow path of the sensor, a sensing structure of the sensor being arranged adjacent to the flow path and being sensitive to the flow rate of that flow, and therefore a sharp distinction between flow sensors and this type of differential pressure sensor is sometimes not even possible. If the electronic sensor is a differential pressure sensor of the flow sensor type, it may have a first and a second sensor port. The first sensor port (or sensor inlet) will then be in fluid-tight communication with the pressure port of the accessory. The second sensor port (or sensor outlet) can be in communication with a reference port, the reference port being arranged such that the air flow does not cause a pressure change at the reference port, or being arranged such that the air flow causes a pressure change of different sign and/or magnitude at the reference port than at the pressure port.

The accessory can further comprise electronic circuitry connected to the electronic sensor, the electronic circuitry being operable to receive sensor signals from the electronic sensor and to derive an output signal based on the sensor signals. The accessory can comprise an output device for creating user feedback based on the output signal. The output device can comprise, e.g., a tone generator for creating acoustic feedback, a visual indicator such as one or more LEDs or an LCD screen for creating visual feedback, and/or a vibration generator for creating tactile feedback to the patient who is using the inhaler.

In some embodiments, the electronic circuitry is configured for wireless communication with a remote device. The remote device can be, for instance, a smartphone, a tablet computer, a notebook computer, or a remote server that is accessible via a wide-area network like the Internet. The electronic circuitry can comprise, for instance, a module enabling a wireless point-to-point link with the remote device, such as a Bluetooth™ module, or a module enabling network communication with the remote device via a wireless link, e.g. via a WiFi™ network or via a cellular network like a GSM network. The electronic circuitry can be configured to transmit the output signal to the remote device via the wireless link. In this manner, real-time feedback about the inhalation process can be provided to the patient, to medical personnel or to a remote analysis engine via the remote device. In addition or in the alternative, inhalation data can be stored in the remote device to be read out later.

The present invention further provides an inhalation system comprising an inhaler having an air inlet for the entry of air into the inhaler and an air outlet for communication with the mouth of a patient, the inhaler being configured such that inhalation by a patient through the air outlet causes an air flow through the inhaler from the air inlet to the air outlet, and further comprising an accessory as described above. The inhaler can in particular be a pMDI, a DPI, a nebulizer or a soft mist inhaler.

In particular, the inhaler can comprise a housing and a drug reservoir received in the housing, the drug reservoir being configured to release an aerosolized drug into the mixing zone when actuated by a patient. In particular, the housing can have an inlet end, the inlet end defining the air inlet of the inhaler, and an outlet end, the outlet end defining the air outlet of the inhaler. The outlet end can be shaped as a mouthpiece. In other embodiments, a separate mouthpiece is provided on the outlet end. The fastening structure of the accessory is advantageously configured to fasten the accessory to the housing of the inhaler. In particular, the fastening structure can be configured to fasten the accessory to the inhaler housing at its inlet end.

The present invention further provides a method of determining a flow rate of an air flow through an inhaler having an air inlet for the entry of air into the inhaler and an air outlet for communication with the mouth of a patient. The method comprises:
fastening to the inhaler an accessory as described above, comprising a pressure port and an electronic sensor in communication with the pressure port, such that the pressure port is arranged upstream from the mixing zone of the inhaler with respect to an air flow that enters the inhaler through the air inlet;
causing an air flow through the inhaler;
measuring a pressure parameter indicative of a pressure change at the pressure port caused by the air flow, using the electronic sensor of the accessory; and
based on the pressure parameter, determining a flow rate parameter that is indicative of the flow rate through the inhaler.

The method can further comprise generating an output signal based on the flow rate parameter. The output signal can be a signal that essentially contains the flow rate parameter itself, or it can depend on the flow rate parameter in some other way. For instance, in a simple case, the output signal can be a binary signal, wherein a logical “1” indicates that the flow rate is within some predetermined limits, whereas a logical “0” indicates that the flow rate is outside these limits. In another embodiment, the output signal can a quantity that is calculated or otherwise derived from the flow rate, such as the total inhaled volume, the peak inspired flow, the airway resistance or other parameters. The method can further comprise generating user feedback based on the output signal, e.g., acoustic, visual and/or tactile user feedback. The method can further comprise transmitting the output signal to a remote device via a wireless link. The method can further comprise operating the remote device to receive the transmitted output signal. The method can further comprise operating the remote device, based on the received output signal, to generate user feedback for the patient.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows, in a highly schematic longitudinal section, an inhalation system comprising an inhaler and an accessory according to a first embodiment, together with a remote control device;

FIG. 2 shows, in a highly schematic longitudinal section, a portion of an inhalation system comprising an inhaler together with an accessory according to a second embodiment;

FIG. 3 shows, in a highly schematic perspective view, an accessory according to a third embodiment;

FIG. 4 shows, in a highly schematic longitudinal section, a portion of the accessory in FIG. 3; and

FIG. 5 shows, in a highly schematic longitudinal section, an inhalation system comprising an inhaler and an accessory according to a fourth embodiment.

### DESCRIPTION OF PREFERRED EMBODIMENTS

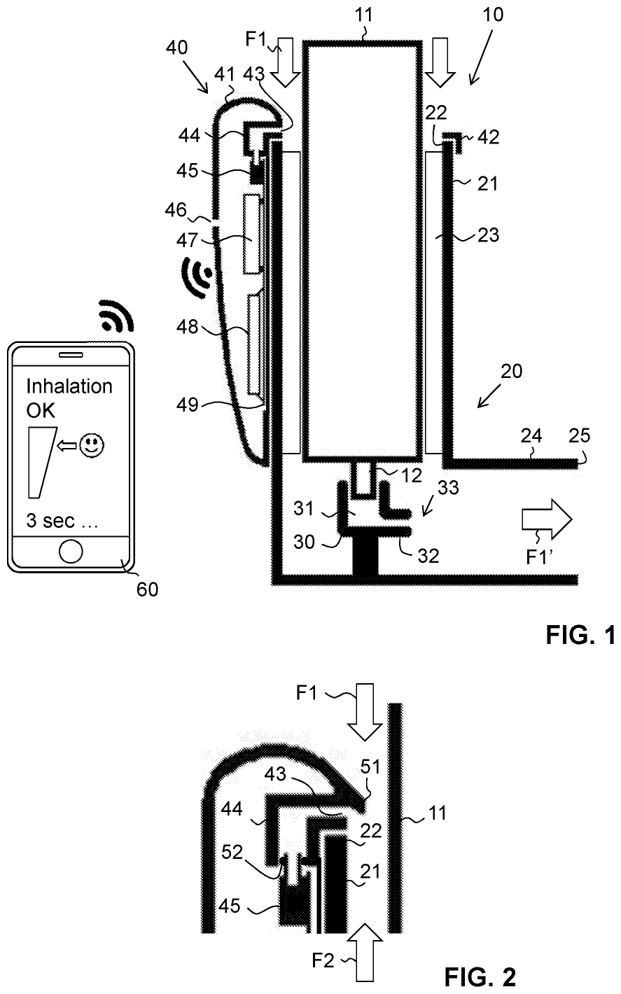

FIG. 1 illustrates, in a highly schematic manner, an inhalation system according to a first embodiment. The inhalation system comprises an inhaler 10 and an accessory 40. The accessory 40 is attached to the inhaler 10 by means of a fastening structure 42 so as to be removable from the inhaler 10.

In the present example, the inhaler 10 is a typical pMDI inhaler. It comprises a generally cylindrical drug reservoir or drug container 11 having at its lower end a metering valve for controlled release of single doses of a drug through a hollow valve stem 12. The metering valve is actuated by pressing the valve stem 12 into drug container 11.

The drug container 11 is received in an inhaler housing 20. The inhaler housing 20 is generally L-shaped. The upright leg of the L forms an upwardly open receiving portion for the drug container 10. The receiving portion has the shape of a hollow cylinder extending upwardly to an upper end 22. The receiving portion is defined by a circumferential housing wall 21. At the open upper end 22 of the receiving portion, the housing wall 21 forms an annular end face. The drug container 11 is held in the receiving portion by a plurality of spacer ribs 23 extending radially inwardly from the housing wall 21. In consequence, the outer circumferential wall of the drug container 11 is spaced from the housing wall 21 of the housing 20, and axially extending air channels are thus formed between the housing wall 21 and the drug container 11.

At the lower end of the receiving portion, the inhaler housing 20 includes a hollow socket 30. The valve stem 12 of the drug container 11 is seated in the socket 30. The socket 30 defines a duct 31 leading from the exit of the valve stem 12 to a nozzle orifice 32. The nozzle orifice 32 opens out into the interior of the inhaler housing 20 in a lateral direction that is transverse to the cylinder axis of the receiving portion, but not necessarily perpendicular to the cylinder axis.

The transverse leg of the L-shaped housing 20 extends in the same lateral direction as the nozzle orifice 32. At its far end, it forms a hollow mouthpiece 24 for insertion into the mouth of a patient. The mouthpiece 24 is laterally open at its end 25. It usually has a flattened cross-sectional shape adapted to the anatomy of a human patient's mouth. This cross-sectional shape is generally different from the cross-sectional shape of the receiving portion.

The open upper end 22 of the receiving portion of the inhaler housing 20 together with the circumferential wall of the drug container 11 forms an air inlet, allowing air to enter the inhaler. The open end 25 of the mouthpiece 24 forms an air outlet. A flow path exists through the inhaler housing 20 between the air inlet and the air outlet. The nozzle orifice 32 is disposed in this air flow path. Downstream from the nozzle orifice 32, the inhaler housing 20 defines a mixing zone 33 for mixing the air flow with the drug released from the drug container 11.

In use, a patient holds the inhaler 10 and applies his mouth to the mouthpiece 24. The patient then inhales through the mouthpiece 24, thereby creating an air flow F1 through the inhaler housing 20 from the air inlet to the air outlet. After the patient has started inhaling through the mouthpiece 14, the drug container 11 is pressed downwardly to release a dose of drug. Due to the pressure in the drug container 11, the drug is propelled through the duct 31 and the nozzle orifice 32 into the mixing zone 33, where it mixes with the air flow to form an aerosol flow F1′, and is inhaled by the patient.

The accessory 40 comprises an accessory housing 41, which is held on the inhaler housing 20 by means of the fastening structure 42. In the present example, the fastening structure 42 forms a ring that is sled onto the open upper end of the receiving portion of the inhaler housing 20. At its upper end, the ring has an inwardly extending flange that rests on the upper end face of the housing wall 21 at the open upper end 22 of the receiving portion, thereby forming an axial stop structure together with the upper end 22 of the receiving portion. This ensures that the accessory housing 41 is fastened to the inhaler housing 20 in a defined axial position.

Inside the accessory housing 41, a carrier 49 in the form of a printed circuit board is disposed. The carrier 49 carries an electronic pressure sensor 45, electronic circuitry 47, and a battery 48. An opening in accessory housing 41 defines a pressure port 43. The electronic pressure sensor 45 communicates with the pressure port 43 pneumatically via a duct 44.

The pressure port 43 is arranged in an inwardly facing wall portion of the accessory housing 41 that is immediately adjacent to the open upper end 22 of the inhaler housing 20. The pressure port 43 is arranged such that the air flow F1, immediately before it enters the inhaler housing 20 at the air inlet, overflows the pressure port 43. The surface of the wall portion of the accessory housing 41 in which the pressure port 43 is arranged is flush with the inside surface of the housing wall 21 of the inhaler housing 20. These surfaces together delimit the air flow F1 at the air inlet. The accessory 40 of the first embodiment does not have any structure that projects into the air flow F1. Thereby it is ensured that the air flow F1 is disturbed as little as possible by the presence of the accessory 40.

In use, the air flow F1 caused by inhalation by the patient causes a negative pressure at the pressure port 43 due to the Bernoulli/Venturi effect caused by the acceleration of the air flow when it enters the inhaler. The magnitude of the negative pressure is directly related to the magnitude of the flow rate of the air flow F1. The negative pressure is registered by the electronic pressure sensor 45. The electronic circuitry 47 reads out the electronic pressure sensor 45 and determines a flow rate parameter from the sensor signals read out from the electronic pressure sensor 45. To this end, the electronic circuitry 47 may employ calibration data that relate measured pressure values to known flow rate values of air flow F1.

Based on the flow rate parameter, the electronic circuitry 47 generates an output signal. The output signal can be, e.g., a digital signal that directly represents the flow rate parameter. In other examples, the output signal can be a digital signal that indicates whether the flow rate parameter is within a predetermined range.

The electronic circuitry 47 can comprise structure for generating user feedback for the patient, based on the output signal. For instance, the electronic circuitry can include a tone generator to generate an audible signal that indicates to the patient whether the flow rate is within a desired range. As another example, the electronic circuitry can include a vibrator to generate a tactile signal in the form of a vibration pattern that indicates to the patient whether the flow rate is within a desired range. As yet another example, the electronic circuitry can include a display, e.g., an LCD display or one or more LEDs, to generate a visual signal that indicates to the patient whether the flow rate is within a desired range.

The electronic circuitry can comprise a wireless communication module for transmitting the output signal via a wireless link to a remote device 60. The remote device 60 can be, e.g., a smartphone, a tablet computer, or a notebook computer. In other embodiments, the remote device can be a dedicated device specifically configured for interaction with the accessory 40. In yet other embodiments, the remote device can be a remote server. The wireless communication module can be, e.g., a Bluetooth™ module for establishing a point-to-point link between the accessory 40 and the remote device 60, or it can be a WiFi™ module for connecting the accessory 40 to a wireless LAN that includes the remote device 60.

In the present example, the remote device 60 executes a computer program (an “app”) that causes the remote device 60 to receive the output signal from the accessory 40 via the wireless link and to generate user feedback using an output device of the remote device. For instance, the app can cause the remote device to display user feedback and/or instructions for correct handling of the inhaler on a display screen of the remote device. In another example, the app can cause the remote device to output an audible signal, e.g., a voice message, via a loudspeaker of the remote device, instructing the user to handle the inhaler in a specific manner. In yet another example, the app can cause the remote device to provide tactile feedback, e.g., by vibrating, depending on the manner in which the patient handles the inhaler. This can all be done in real time.

In addition, the app can cause the remote device to store the received output signals for later readout, and/or to transmit the received output signals or quantities derived from the received output signals, such as statistical data, to a remote server for analysis. This enables the monitoring of the usage of the inhaler by medical personnel. In other embodiments, the output signals are directly transmitted from the attachment to a remote server for analysis.

The electronic pressure sensor 45 can, in particular, be a differential pressure sensor that is based on a flow measurement principle. A differential pressure sensor of this type has two sensor ports: a sensor inlet and a sensor outlet. A pressure difference between the sensor inlet and the sensor outlet causes a sensor gas flow through a sensor flow channel defined inside the differential pressure sensor. A flow-sensitive structure is arranged adjacent to the sensor flow channel for measuring a flow rate of the sensor gas flow through the sensor flow channel. Thus, a differential pressure sensor of this type essentially acts as a flow sensor that is configured to determine the pressure difference based on the determination of a flow rate through a flow channel between the sensor ports.

A suitable flow sensor that can be used to determine differential pressure is disclosed, e.g., in US 2016/0161314 A1. The inlet and outlet tubes of the flow sensor disclosed in this document can act as the sensor ports referred to in the present disclosure.

If a differential pressure sensor or flow sensor is used, one of the sensor ports of the sensor is pneumatically connected to the pressure port 43 in a fluid-tight manner. The other sensor port advantageously is pneumatically connected to the environment of the accessory 40 in a region that is not influenced by the air flow F1. To this end, the accessory housing 41 can comprise a reference port 46 in the form of an opening, the opening being arranged in a region of the accessory housing 41 that faces away from the inhaler. The opening causes the pressure inside the accessory housing 41 to be equal to the pressure of the environment of the accessory 40 in a region that is unaffected by the air flow F1. The sensor port that is not connected to the pressure port 43 can therefore be simply open towards the inside of accessory housing 41, without a fluid-tight connection being required between this sensor port and the reference port 46.

It should be noted that the accessory 40 of the first embodiment cannot distinguish between a flow F1 caused by inhalation through the inhaler and a reverse flow in the opposite direction, as it would be caused by exhalation through the inhaler.

FIG. 2 schematically illustrates a second embodiment that avoids this disadvantage. The second embodiment is largely identical to the first embodiment, and only a portion around the upper open end of the inhaler housing 20 is illustrated. Elements that have the same functionality as in the first embodiment are designated with the same reference signs as in FIG. 1.

A key difference of the second embodiment as compared to the first embodiment lies in the design of the accessory housing 41 in the immediate vicinity of the pressure port 43. Whereas in the first embodiment the pressure port 43 is arranged in a smooth wall portion of the accessory housing 41 that modifies the air flow F1 only minimally, in the second embodiment the accessory housing 41 comprises a flow-modifying structure 51 that deliberately projects into the flow path of the air flow F1 to modify the air flow. In the present example, the flow-modifying structure 51 acts as a local barrier immediately downstream from the pressure port 43 for a reverse air flow F2, thereby creating a positive dynamic pressure (velocity pressure) at the pressure port 43 for the reverse air flow F2. In this manner, the air flow F1 due to inhalation and the reverse air flow F2 due to exhalation can be readily distinguished by the sign of the pressure change at the pressure port 43.

Also illustrated in FIG. 2 is the fluid-tight connection between the electronic sensor 45 and the duct 44 that leads to the pressure port 43, symbolized by a gasket 52.

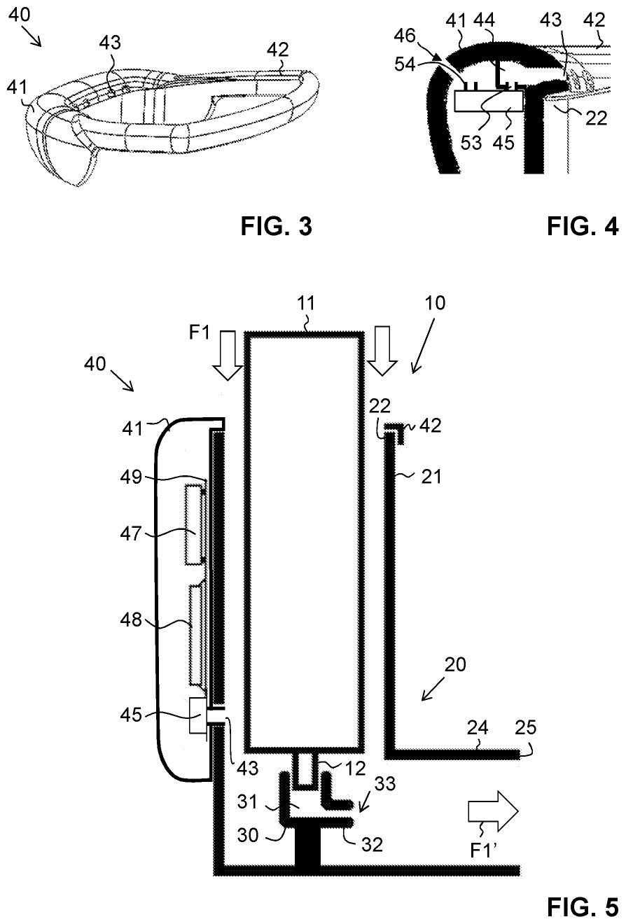

FIGS. 3 and 4 illustrate a third embodiment of an accessory 40. Again, elements that have the same functionality as in the first embodiment are designated with the same reference signs as in FIG. 1.

As in the first and second embodiments, the fastening structure 42 is designed as a ring for attachment to the inlet end of the inhaler, more specifically, to the upper end 22 of the receiving portion of the inhaler housing. As in the first and second embodiments, the accessory housing 41 forms an axial stop with the upper end face of the inhaler housing to define the correct axial position of the accessory 40 on the inhaler housing. By the design of the fastening structure, the accessory 40 can only be attached to the inlet end of the inhaler, whereas it is impossible to accidentally fasten the accessory 40 to the outlet end, i.e., to the mouthpiece of the inhaler, due to its different shape.

The housing 41 of the accessory 40 according to the third embodiment has a plurality of openings, in particular, four openings, arranged along the circumference of the open end 22 of the receiving portion of the inhaler housing. As apparent from FIG. 4, all openings communicate pneumatically with a common duct 44, which in turn communicates pneumatically with the electronic sensor 45. In this manner, all openings together form the pressure port 43 of the third embodiment. Each opening has a sufficiently small individual cross-sectional area that dust or drops of liquid cannot easily enter the duct 44. At the same time, the openings together have a total cross-sectional area that gas exchange is possible between the inside and the outside of the duct 44 at a sufficiently high rate for ensuring proper operation of the electronic sensor 45.

As apparent from the schematic representation in FIG. 4, the electronic sensor 45 can be a differential pressure sensor or flow sensor, having two sensor ports 53, 54, the first sensor port being connected in a fluid-tight manner to the duct 44, while the second sensor port is in pneumatic communication with the environment via the reference port 46.

An accessory according to the third embodiment has been tested in conjunction with a commercially available pMDI. Measurements showed that an inhalation air flow of 601/min caused a pressure difference of about 20 Pa between the two sensor ports. A differential pressure of this magnitude can be readily detected by commercially available differential pressure sensors.

FIG. 5 illustrates a fourth embodiment of an accessory 40, together with a correspondingly configured inhaler 10. Again, elements that have the same functionality as in the first embodiment are designated with the same reference signs as in FIG. 1.

As in the first to third embodiments, the accessory 40 is attached to the outside of the inhaler housing 20 and can be easily removed from the inhaler. In the fourth embodiment, the circumferential wall 21 of the inhaler housing 20 is provided with a through-hole upstream from nozzle orifice 32, and the pressure port 43 is formed by the open end of a short pipe that extends into this through-hole to measure the flow rate of the air flow F1 without significantly disturbing the air flow. By arranging the pressure port 43 upstream of the nozzle orifice 32 with respect to the inhalation air flow F1, it is ensured that the electronic sensor 45 cannot be contaminated with the drug that is released through the nozzle orifice as long as the inhaler is used properly.

In a modification of the fourth embodiment (not illustrated in the drawings), the accessory 40 or the inhaler housing 20 is provided with a flow-modifying structure adjacent to the pressure port 43 inside the inhaler housing 20 in order to be able to distinguish between inhalation and exhalation, as discussed above in conjunction with the second embodiment.

While the accessory of the first to third embodiment can be fitted to any existing inhaler, the inhaler of the fourth embodiment may require adaptation to the intended use by providing the inhaler housing 20 with the through-hole for the pressure port 43.

While exemplary embodiments of the invention have been illustrated with reference to the drawings, the invention is by no means limited to these embodiments, and many modifications are possible without departing from the scope of the present invention. For instance, the electronic sensor can be of a different type than described above. In particular, the electronic sensor can be any other type of pressure sensor, for instance an absolute pressure sensor or a relative pressure sensor that is based on a different measurement principle than a flow measurement. In particular, the pressure sensor can be a barometric pressure sensor. The accessory can be fastened to the inhaler in a different manner than described. For instance, the accessory can be clamped to the inhaler body by two prong-like arms. The accessory of the present invention can also be used with other types of inhalers than the pMDI shown.

1.-15. (canceled)

16. An accessory for an inhaler, the inhaler comprising an inhaler housing having a housing wall, the inhaler having an air inlet for the entry of air into the inhaler and an air outlet for communication with the mouth of a patient, such that inhalation by a patient through the air outlet causes an air flow through the inhaler from the air inlet to the air outlet, the inhaler defining a mixing zone located between the air inlet and the air outlet for mixing the air flow with a drug, the accessory comprising:
an accessory housing, the accessory housing defining an axial stop structure configured to abut to a proximal end face of the housing wall of the inhaler housing at the air inlet of the inhaler when the accessory is fastened to the inhaler, the accessory housing having a wall portion adjacent to the stop structure, configured to be essentially flush with the housing wall of the inhaler housing when the accessory is fastened to the inhaler, the wall portion and the housing wall of the inhaler housing delimiting a flow path of the air flow;
a pressure port, the pressure port being formed by one or more openings in the wall portion of the accessory housing which is configured to be essentially flush with the housing wall of the inhaler housing, the pressure port being configured to be arranged upstream from the air inlet of the inhaler with respect to the air flow; and
an electronic sensor for determining a flow rate of the air flow, the electronic sensor being received in the accessory housing, the electronic sensor being in communication with the pressure port, the electronic sensor being sensitive to a pressure change at the pressure port caused by the air flow.

17. The accessory of claim 16, wherein the pressure port is formed by a plurality of openings in the accessory housing, the openings being distributed circumferentially along a circumference of the air inlet of the inhaler when the accessory is fastened to the inhaler.

18. The accessory of claim 16, further comprising a fastening structure for fastening the accessory to the inhaler.

19. The accessory of claim 18, wherein the fastening structure comprises a ring configured to be arranged around the inhaler housing.

20. The accessory of claim 16, wherein the accessory is configured to modify the air flow adjacent to the pressure port in such a manner that a reverse air flow due to exhalation by the patient through the inhaler causes a pressure change at the pressure port with opposite sign as compared to the pressure change caused by the air flow due to inhalation.

21. An inhalation system comprising an inhaler and an accessory, the accessory being fastened to the inhaler,
the inhaler comprising an inhaler housing having a housing wall, the inhaler having an air inlet for the entry of air into the inhaler and an air outlet for communication with the mouth of a patient, such that inhalation by a patient through the air outlet causes an air flow through the inhaler from the air inlet to the air outlet, the inhaler defining a mixing zone located between the air inlet and the air outlet for mixing the air flow with a drug,
the accessory comprising:
an accessory housing, the accessory housing having a wall portion adjacent to the air inlet of the inhaler, the wall portion being essentially flush with the housing wall of the inhaler housing, the wall portion and the housing wall of the inhaler housing delimiting a flow path of the air flow;
a pressure port, the pressure port being formed by one or more openings in the wall portion of the accessory housing which is essentially flush with the housing wall of the inhaler housing, the pressure port being arranged upstream from the air inlet of the inhaler with respect to the air flow; and
an electronic sensor for determining a flow rate of the air flow, the electronic sensor being received in the accessory housing, the electronic sensor being in communication with the pressure port, the electronic sensor being sensitive to a pressure change at the pressure port caused by the air flow.

22. The inhalation system of claim 21, wherein the accessory housing defines an axial stop structure, the axial stop structure abutting to a proximal end face of the housing wall of the inhaler housing at the air inlet of the inhaler, and wherein the wall portion of the accessory housing in which the pressure port is formed and which is essentially flush with the housing wall of the inhaler housing is arranged adjacent to the axial stop structure.

23. The inhalation system of claim 21, wherein the pressure port is formed by a plurality of openings in the accessory housing, the openings being distributed circumferentially along a circumference of the air inlet of the inhaler.

24. The inhalation system of claim 21, wherein the accessory comprises a fastening structure for fastening the accessory to the inhaler.

25. The inhalation system of claim 24, wherein the fastening structure comprises a ring that is arranged around the inhaler housing.

26. The inhalation system of claim 21, wherein the accessory is configured to modify the air flow adjacent to the pressure port in such a manner that a reverse air flow due to exhalation by the patient through the inhaler causes a pressure change at the pressure port with opposite sign as compared to the pressure change caused by the air flow due to inhalation.

27. The inhalation system of claim 21, wherein the inhaler comprises a drug reservoir received in the housing, the drug reservoir being configured to release an aerosolized drug into the mixing zone.

28. An accessory for an inhaler, the inhaler having an air inlet for the entry of air into the inhaler and an air outlet for communication with the mouth of a patient, such that inhalation by a patient through the air outlet causes an air flow through the inhaler from the air inlet to the air outlet, the inhaler defining a mixing zone between the air inlet and the air outlet for mixing the air flow with a drug, the accessory comprising:
an accessory housing;
a pressure port, the pressure port being arranged upstream from the air inlet of the inhaler with respect to the air flow when the accessory is fastened to the inhaler, the pressure port being formed by a plurality of openings in the accessory housing, the openings being distributed circumferentially along a circumference of the air inlet of the inhaler when the accessory is fastened to the inhaler;
an electronic sensor for determining a flow rate of the air flow, the electronic sensor being received in the accessory housing, the electronic sensor being in communication with the pressure port, the electronic sensor being sensitive to a pressure change at the pressure port caused by the air flow.

29. The accessory of claim 28, further comprising a fastening structure for fastening the accessory to the inhaler.

30. The accessory of claim 29, wherein the fastening structure comprises a ring configured to be arranged around an inhaler housing of the inhaler.

31. The accessory of claim 28, wherein the accessory is configured to modify the air flow adjacent to the pressure port in such a manner that a reverse air flow due to exhalation by the patient through the inhaler causes a pressure change at the pressure port with opposite sign as compared to the pressure change caused by the air flow due to inhalation.

32. An inhalation system comprising an inhaler and an accessory, the accessory being fastened to the inhaler,
the inhaler having an air inlet for the entry of air into the inhaler and an air outlet for communication with the mouth of a patient, such that inhalation by a patient through the air outlet causes an air flow through the inhaler from the air inlet to the air outlet, the inhaler defining a mixing zone between the air inlet and the air outlet for mixing the air flow with a drug,
the accessory comprising:
an accessory housing;
a pressure port, the pressure port being arranged upstream from the air inlet of the inhaler with respect to the air flow, the pressure port being formed by a plurality of openings in the accessory housing, the openings being distributed circumferentially along a circumference of the air inlet of the inhaler; and
an electronic sensor for determining a flow rate of the air flow, the electronic sensor being received in the accessory housing, the electronic sensor being in communication with the pressure port, the electronic sensor being sensitive to a pressure change at the pressure port caused by the air flow.

33. The inhalation system of claim 32, wherein the accessory comprises a fastening structure for fastening the accessory to the inhaler.

34. The inhalation system of claim 33, wherein the fastening structure comprises a ring configured to be arranged around the inhaler housing.

35. The inhalation system of claim 32, wherein the accessory is configured to modify the air flow adjacent to the pressure port in such a manner that a reverse air flow due to exhalation by the patient through the inhaler causes a pressure change at the pressure port with opposite sign as compared to the pressure change caused by the air flow due to inhalation.

36. The inhalation system of claim 32, wherein the inhaler comprises a drug reservoir received in the housing, the drug reservoir being configured to release an aerosolized drug into the mixing zone.
